(19) United States
(12) Patent Application Publication
(10) Pub. No.: US 2026/0154951 A1
(43) Pub. Date: Jun. 4, 2026

(54) SYSTEMS AND METHODS FOR ACCESSORY LOCALIZATION TO DRIVE IMAGING WORKFLOWS

(71) Applicant: GE Precision Healthcare LLC, Waukesha, WI (US)

(72) Inventors: Deepa Anand, Bengaluru (IN); Dattesh Shanbhag, Bengaluru (IN); Mahendra Patil, Bengaluru (IN); Krishna Seetharam Shriram, Bengaluru (IN); Sajith Rajamani, Bengaluru (IN); Jessica Buzek, Waukesha, WI (US); Sai Gannavarappu, Bangalore (IN)

(73) Assignee: GE Precision Healthcare LLC, Waukesha, WI (US)

(21) Appl. No.: 18/969,056

(22) Filed: Dec. 4, 2024

(51) Int. Cl. G06V 10/778; A61B 6/00; G06V 10/74; G06V 10/75; G06V 10/764; G06V 10/77; G06V 10/94; G16H 30/20; G16H 40/63

(52) U.S. Cl. CPC G06V 10/7788; A61B 6/5247; A61B 6/563; G06V 10/751; G06V 10/761; G06V 10/764; G06V 10/7715; G06V 10/945; G16H 30/20; G16H 40/63; G06V 2201/03

(57) ABSTRACT

A medical imaging system includes an image library comprising a plurality of library images depicting known objects and a memory device storing instructions thereon that, when executed, cause a processing circuit to: receive, via a camera of the medical imaging device, a patient image within an imaging space, detect, using a trained model, an object in the patient image, classify, using the trained model, the detected object as either one of a known object from the image library or an unknown object, display an indication of the detected object on a user interface, receive, via the user interface, a user input indicating whether the detected object has been correctly classified as a known object or an unknown object by the trained model, and at least one of obtain a medical image of the patient and the detected object or update the image library based on the user input.

### FIELD

Embodiments of the subject matter disclosed herein relate to medical imaging, and more particularly, to improved object detection during a medical imaging process.

### BACKGROUND

During a medical imaging workflow, a plurality of medical images of a patient are obtained by a technician to measure or detect various aspects of anatomical features present within the medical images. These images are subsequently analyzed by a clinician to observe a condition or to identify any abnormalities.

### SUMMARY

One embodiment relates to a medical imaging system. The medical imaging system includes a medical imaging device configured to transmit electromagnetic pulse signals to a patient within an imaging space having a magnetostatic field formed therein to obtain medical images of the patient, an image library including a plurality of library images depicting known objects, and a processing circuit having a processor coupled to a memory device storing instructions thereon that, when executed, cause the processing circuit to: receive, via a camera of the medical imaging device, a patient image of a patient within the imaging space, detect, using a trained model, an object in the patient image, classify, using the trained model, the detected object as either one of a known object from the image library or an unknown object, display an indication of the detected object on a user interface of the medical imaging device, receive, via the user interface, a user input indicating whether the detected object has been correctly classified as a known object or an unknown object by the trained model, and at least one of obtain a medical image of the patient and the detected object or update the image library based on the user input.

Another embodiment relates to a medical imaging system. The medical imaging system includes a medical imaging device configured to transmit electromagnetic pulse signals to a patient within an imaging space having a magnetostatic field formed therein to obtain medical images of the patient, an image library including a plurality of library images depicting known objects, a camera associated with the medical imaging device, configured to receive a patient image of a patient within the imaging space, a trained model configured to: detect an object in the patient image and classify the detected object as either one of a known object from the image library or an unknown object, a user interface associated with the medical imaging device configured to display an indication of the detected object, receive a user input indicating whether the detected object has been correctly classified as a known object or an unknown object by the trained model, and at least one of obtain a medical image of the patient and the detected object or update the image library based on the user input.

Another embodiment relates to a method. The method includes receiving, by a processing circuit, via a camera of a medical imaging device, a patient image of a patient within an imaging space, detecting, by the processing circuit, using a trained model, an object in the patient image, classifying, by the processing circuit, using the trained model, the detected object as either one of a known object from an image library or an unknown object, displaying, by the processing circuit, an indication of the detected object on a user interface of the medical imaging device, receiving, by the processing circuit, via the user interface, a user input indicating whether the detected object has been correctly classified as a known object or an unknown object by the trained model, and at least one of obtaining, by the processing circuit, a medical image of the patient and the detected object or updating, by the processing circuit, the image library based on the user input.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of a medical imaging system, according to an example embodiment.

FIG. 2 is a flow chart illustrating a method of detecting an object using a model, according to an example embodiment.

FIG. 3 is a flow chart illustrating a method of detecting an object using a model, according to an example embodiment.

FIG. 4A is a first flow chart illustrating a method of detecting an object during a medical imaging workflow, according to an example embodiment.

FIG. 4B is a second flow chart illustrating a method of detecting an object during a medical imaging workflow, according to an example embodiment.

FIG. 4C is a third flow chart illustrating a method of detecting an object during a medical imaging workflow, according to an example embodiment.

FIG. 5A is a first flow chart illustrating a method of user validation of object detection during a medical imaging workflow, according to an example embodiment.

FIG. 5B is a second flow chart illustrating a method of user validation of object detection during a medical imaging workflow, according to an example embodiment.

FIG. 6A is a flow chart illustrating a method of training a model to detect objects during a medical imaging workflow, according to an example embodiment.

FIG. 6B is a flow chart illustrating a method of training a model to detect objects during a medical imaging workflow, according to an example embodiment.

FIG. 7 depicts test images for training a model to detect objects using the methods of FIGS. 2-6B, according to an example embodiment.

FIG. 8 depicts detected objects using the methods of FIGS. 2-6B, according to an example embodiment.

FIG. 9 depicts a plurality of objects and templates used in detecting an object during a medical imaging workflow, according to an example embodiment.

FIG. 10 depicts a flow chart illustrating a method of detecting an object during a medical imaging workflow, according to an example embodiment.

FIG. 11 depicts a flow chart illustrating a method of object detection, according to an example embodiment.

### DETAILED DESCRIPTION

Referring generally to the figures, artificial intelligence (AI) systems and methods for performing object detection to improve imaging workflows are disclosed. The systems and methods disclosed herein use various processing circuits, such as an imaging processing circuit, to identify objects, particularly imaging accessories such as magnetic resonance (MR) coils or electrocardiogram (ECG) leads, in an image of a patient. The systems and methods use models, such as deep learning models, and an image library to detect objects and classify them as being objects of interest (e.g., imaging accessories) or objects of non-interest (e.g., objects that are not imaging accessories). Further, a technician may review the classification performed by the model to confirm model performance and classification.

Currently, cameras are increasingly being utilized in imaging workflows, such as MR imaging workflows, to simplify the imaging process and assist untrained imaging technicians. Various medical imaging workflows, such as MR scanning, utilize accessories, such as MR coils, that are placed on the patient being scanned. Detection of these imaging accessories is important, as the accessories are placed on the patient at the location where the medical scan/image should be taken. Thus, it may be important to automatically detect these imaging accessories, particularly to improve automation of overall imaging workflow.

Current AI methods and models exist for object detection in images. However, the models currently deployed are trained in a supervised manner and can detect accessories that the models have already been trained on. However, current models may fail to detect imaging accessories when the accessories have been generalized compared to the training data, such as when an imaging accessory has changed in shape, color, texture, etc. Further, current models may be unable to detect different types of accessories that the model has not been trained on. In order for current models to be able to detect different or generalized accessories, the model may be required to be continually retrained with additional and/or different images. This may cause increased computer processing power devoted to training and deploying the models.

The systems and methods described herein are directed to a template-based AI model that is trained and deployed once. The model is used in combination with an image library that stores images or templates of different imaging accessories expected to be seen on patients. The image library is updated, either by the model or a technician, as images showing new or different imaging accessories are introduced. Updating the image library as opposed to retraining the model may reduce computing processing times, computer processing power, and may increase available computer memory that would have otherwise been reserved for processes used in continually retraining the model.

When the model receives an image of a patient, the model uses a support set of images from the image library that includes example or template images of accessories to identify accessories in the image of the patient. Additionally, the model uses a support set of images from the image library that includes accessory mimics (e.g., objects visually similar to accessories) to reduce instances of the model returning false positive results.

The systems and methods further include a reinforcement step by way of utilizing a feedback loop and/or an imaging technician or other user to confirm the objects detected by the model. For example, the technician may view a patient of an image that includes indications of objects that the model has detected and classified as imaging accessories. The technician can confirm accurate detections made by the model, as well as identify any false negative and/or false positive identifications. When the model has detected a false positive (e.g., the model has incorrectly identified a non-accessory as an accessory), the incorrectly identified object is added to the accessory mimic support set in the image library. Any accessory not identified by the model is marked by the technician and is added to the accessory support set in the image library.

The systems and methods described herein provide a way of continually improving object detection by the model without devoting computer resources and time to retraining the model. Using the template library in conjunction with technician feedback, the model does not need to be retrained, thereby avoiding problems associated with retraining, such as catastrophic forgetting and multiple model deployment. The systems and methods also enable a sustained ability to detect imaging accessories in patient images, which may assist untrained technicians in placing imaging accessories on a patient in preparation for a medical scan or image. Further, the systems and methods allow the model to adapt to site-specific changes in imaging accessory designs. The systems and methods may also be used to detect potential safety hazards, such as IV lines caught on an imaging table, an imaging accessory being misplaced, cables crossed over, etc. Currently, these processes, such as hazard detection, may be performed manually, which increases an amount of time spent taking medical images/scans, and may lead to increased instances of safety events. Automatic object detection may therefore eliminate manual intervention, creating faster and safter working conditions and environments.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

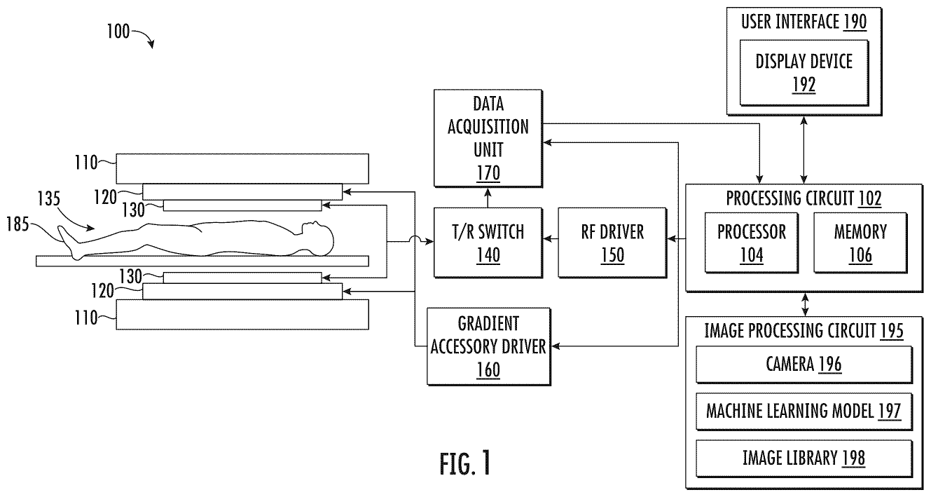

Referring to FIG. 1, a block diagram of a medical imaging system 100 is shown according to an example embodiment. The medical imaging system 100 is configured to obtain medical images of a patient. As shown in FIG. 1, the medical imaging system 100 is a magnetic resonance (MR) imaging system. However, it will be appreciated that the medical imaging system 100 can be any type of medical imaging system. For example, the medical imaging system 100 can be or include at least one of an MR imaging device, a computed tomography (CT) imaging device, a positron emission tomography (PET) imaging device, a single-photon emission computed tomography (SPECT) imaging device, or an X-ray imaging device, among others.

The medical imaging system 100 includes a processing circuit 102 including a processor 104 and a memory 106, a magnetostatic field magnet 110, a gradient coil 120, an RF body coil 130, a transmit/receive (T/R) switch 140, an RF driver 150, a gradient coil driver 160, a data acquisition unit 170, a patient bed 180 for a patient 185, and a user interface 190 including a display device 192. It will be appreciated that the processing circuit 102 and the user interface 190 can be separate from the medical imaging system 100, and the medical imaging system 100 may instead include its own dedicated processing circuit and user interface.

The medical imaging system 100 is configured to transmit electromagnetic pulse signals to the patient 185 when the patient 185 is on the patient bed 180 within an imaging space 135 having a magnetostatic field formed therein to scan the patient 185 as part of a medical imaging process. The medical imaging system 100 is operable to obtain magnetic resonance (MR) signals from the patient 185 to construct an image of a slice of the patient 185 from a series of images based on the MR signals obtained through the medical imaging process.

The processing circuit 102 includes a processor 104 and memory 106 which are configured to carry out the functions of the medical imaging system 100. The processor 104 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 104 may be shared by multiple circuits. Alternatively or additionally, the processor 104 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

In some embodiments, the processing circuit 102 may include multiple processors configured to perform the processing operations/functionality described with reference to processor 104. It should be appreciated that other embodiments may use a different arrangement of processors. The processor 104 may be in electronic communication with the user interface 190 and the display device 192 such that the processor 104 may process data and generate images or other information to display on the display device 192.

The memory 106 may be configured to, for example, store processed or unprocessed volumes of data obtained by the medical imaging system 100 (e.g., image data). For example, the memory 106 may be a hospital picture archiving and communication system (PACS). The memory 106 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 106 may be or include tangible, non-transient volatile memory or non-volatile memory. The memory 106 may also include database components, object code components, script components, or any other type of information structure for supporting the activities and information structures described in the present application.

In various embodiments, the memory 106 may have varying capacity (e.g., storage space) across embodiments of the medical imaging system 100. For example, the memory 106 may be configured to store sensor data obtained over several days or years of operation of the medical imaging system 100. The sensor data may be stored in the memory 106 such that the sensor data may be retrieved according to an order/time of acquiring the data. That is, the sensor data may be stored with a timestamp indicating a time at which the sensor data was collected and may be retrieved starting with an oldest time at which the sensor data was collected.

The magnetostatic field magnet 110 includes an annular superconducting magnet mounted within a toroidal vacuum vessel. The magnet defines a cylindrical space surrounding the patient 185, and generates a constant primary magnetostatic field.

The gradient coil 120 generates a gradient magnetic field within the imaging space 135, providing three-dimensional positional information for the magnetic resonance signals received by RF coil arrays (not shown). The gradient coil 120 includes three gradient coil systems, each producing a gradient magnetic field along one of the three mutually perpendicular spatial axes. These fields are applied in the frequency encoding direction, phase encoding direction, and slice selection direction based on the imaging requirements. Specifically, the gradient coil 120 creates a gradient field along the slice selection (or scan) direction to select the desired slice of the patient 185, and the RF body coil 130 or local RF coil arrays transmit an RF pulse to that slice. The gradient coil 120 also generates a gradient field in the phase encoding direction of the patient 185 to phase encode the magnetic resonance signals from the excited slice, and then applies a gradient field in the frequency encoding direction of the patient 185 to frequency encode the magnetic resonance signals from that slice.

The RF coil arrays enclose the region to be imaged of the patient 185. The RF coil arrays can transmit an RF pulse comprising an electromagnet waive to the patient 185 to generate a high frequency magnetic field. The RF coil arrays transmit the RF pulse in the imaging space 135 based on a signal received from the processing circuit 102. The high frequency magnetic field excites a spin of protons in the slice to be imaged of the patient 185. The RF coil arrays receive, as a MR signal, the electromagnetic wave generated when the proton spin returns into alignment with an initial magnetization vector. In some embodiments, each RF coil can transmit and receive an RF pulse using the same RF coil. In some embodiments, the RF coil may only receive MR signals, and not transmit the RF pulse. Different RF coil arrays may be utilized for different scanning objectives. Accordingly, one or more of the RF coil arrays may be disconnected from the medical imaging system 100 so that a different coil array may be connected to the medical imaging system 100. The RF coil arrays may be coupled to the RF driver 150 and the data acquisition unit 170 via the T/R switch 140.

The RF body coil 130 encloses the imaging space 135 and generates RF magnetic field pulses orthogonal to a main magnetic field produced by the magnetostatic field magnet 110 within the imaging space 135 to excite the nuclei of the patient 185. The RF body coil 130 is fixed to the medical imaging system 100. In some embodiments, the RF body coil 130 has a larger coverage area than the RF coil arrays and can be used to transmit or receive signals to the whole body of the patient 185. In some embodiments, using receive-only RF coil arrays and transmit body coils, the medical imaging system 100 can provide a uniform RF excitation and good image uniformity at the expense of high RF power provided to the patient 185. In some embodiments, using a transmit-receive RF coil array, the medical imaging system 100 provides the RF excitation to the region of interest and receives the MR signal, thereby decreasing the RF power provided to the patient 185. The RF body coil 130 can be configured to operate in a transmit-only mode, a receive-only mode, or a transmit-receive mode. The RF coil arrays can be configured to operate in a transmit-receive mode or a receive-only mode.

The T/R switch 140 is configured to selectively connect the RF body coil 130 to the data acquisition unit 170 when operating in a receive mode, and to the RF driver 150 when operating in a transmit mode. The T/R switch 140 can selectively electrically connect one or more of the RF coil arrays to the data acquisition unit 170 when the RF coil arrays operate in the receive mode, and to the RF driver 150 when RF coil arrays operate in the transmit mode. In some embodiments, when the RF coil arrays and the RF body coil 130 are both used in a single scan and the RF coil arrays are configured to receive MR signals and the RF body coil 130 is configured to transmit RF signals, the T/R switch 140 is configured to direct control signals from the RF driver 150 to the RF body coil 130 and to direct received MR signals from the RF coil arrays to the data acquisition unit 170.

The RF driver 150 includes a gate modulator, an RF power amplifier, and an RF oscillator configured to drive the RF coil arrays and create a high-frequency magnetic field in the imaging space 135. The RF driver 150 is configured to modulate, based on a control signal from the processing circuit 102 and using the gate modulator, the RF signal received from the RF oscillator into a signal of predetermined timing having a predetermined envelope. The RF power amplifier is configured to amplify the RF signal modulated by the gate modulator and then the modulated RF signal is output to the RF coil arrays.

The gradient coil driver 160 is configured to drive the gradient coil 120 based on a control signal received from the processing circuit 102 and thereby generate a gradient magnetic field in the imaging space 135. The gradient coil driver 160 includes three driver circuits that correspond to each of the three gradient coil systems of the gradient coil 120.

The data acquisition unit 170 includes a preamplifier, a phase detector, and an analog/digital converter configured to acquire the MR signals received by the RF coil arrays. The phase detector is configured to phase detect, using an output from the RF oscillator of the RF driver 150 as a reference signal, the MR signals received from the RF coil arrays and amplified by the preamplifier. The phase detector is configured to output a phase-detected analog magnetic resonance signals to the analog/digital converter for conversion into digital signals. The digital signals are then output to the processing circuit 102.

The patient bed 180 is a table or other surface configured to support the patient 185. The patient can be placed on the patient bed 180 and then moved into the imaging space 135 of the medical imaging system 100. The patient bed 180 may selectively move, with the patient 185 disposed on the patient bed 180, into and out of the imaging space 135 based on control signals received from the processing circuit 102. In some embodiments, one or more RF coil arrays are coupled to the patient bed 180 and move with the patient 185.

The processing circuit 102 is configured to control the operations of the medical imaging system 100 based on user inputs received via the user interface 190. For example, based on a user input, the processing circuit 102 can control the various parts of the medical imaging system 100 (e.g., the patient bed 180, the RF driver 150, gradient coil driver 160, and data acquisition unit 170) to carry out operations to perform a predetermined medical imaging process on the patient 185. The processing circuit 102 can carry out all processes described herein with respect to processing circuit 102.

The user interface 190 can include any type of control elements configured to enable an operator or technician of the medical imaging system 100 to interact with the medical imaging system 100 and to enter commands to control the medical imaging system 100.

The user interface 190 may be used by an operator of a medical imaging system (e.g., a technician or clinician), such as the medical imaging system 100. For example, an operator of the medical imaging system 100 may use the user interface 190 to control the input of patient data, to change a scanning or display parameter, and/or to select various other modes, operations, parameters, etc. of the medical imaging system 100. In some embodiments, the user interface 190 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 190 may be an electronic device that was designed and developed for use in a medical environment or vehicle environment.

According to some embodiments, the user interface 190 may be physically separate from the rest of the medical imaging system 100. The user interface 190 may communicate with the processor 104 through a wireless protocol, such as Wi-Fi, Bluetooth, wireless local area network (WLAN), near-field communication, and so on. According to some embodiments, the user interface 190 may communicate with the processor 104 through an application programming interface (API).

In some embodiments, the user interface 190 may include physical controls such as one or more of buttons, sliders, a rotary knob, a mouse, a keyboard, a trackball, a steering wheel, hard keys linked to specific actions, soft keys that may be configured to control different functions, and so on. In some embodiments, the user interface includes a speaker or a microphone. As shown in FIG. 1, the user interface 190 may also include a display device 192. In some embodiments, the display device 192 may be configured to display a graphical user interface (GUI) based on an instruction from the memory 106. The GUI may include user interface icons representing commands and instructions relating to the operation of the medical imaging system 100. The user interface icons of the GUI may be configured such that a user may select a specific user interface icon in order to initiate a specific function controlled by the GUI. For example, various user interface icons may be used to represent windows, menus, buttons, cursors, scroll bars, and so on. That is, the physical controls of the user interface 190 may be included as individual hardware elements, as user interface icons displayed on the display device 192, or as a combination of hardware elements and user interface icons.

In some embodiments, the display device 192 may include a touch-sensitive display device or a touch screen. According to such embodiments, the touch screen may be configured to interact with the GUI displayed by the display device 192 such that a user can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 192 or may be separate from the display device 192. The user interface 190 may also include a proximity sensor configured to detect objects and/or gestures that are within a predetermined distance (e.g., five feet, six inches, ten centimeters, etc.) of the proximity sensor. In various embodiments, the proximity sensor may be located on the display device 192 or as part of a touch screen that is separate from the display device 192.

The medical imaging system 100 further includes an image processing circuit 195. The image processing circuit 195 may include a camera 196, a machine learning model 197, and an image library 198. The image processing circuit 195 may be configured to detect objects within the imaging space 135. Specifically, the image processing circuit 195 may be configured to detect accessories used during the imaging process performed by the medical imaging system 100. For example, when an MR imaging is performed on the patient 185, one or more imaging accessories (e.g., one or more RF coils) may be used to facilitate imaging. For example, an RF body coil 130 may be placed on or around the patient 185 at a location or proximate a location of the patient site to be imaged. In some embodiments, (e.g., in implementations where the medical imaging system 100 is an imaging system other than a MR imaging system), the accessories may be, for example, ECG leads. The image processing circuit 195 may utilize or include a machine learning model (e.g., a deep learning model) that is trained to detect objects located on the patient 185 or otherwise in the imaging space 135. The image processing circuit 195 may facilitate placement of accessories on the patient 185 and/or may facilitate adaptation to site-specific changes in accessory designs.

The camera 196 may be configured to capture or facilitate capturing an image of the patient. In some embodiments, the camera 196 may be positioned proximate the imaging space 135 to capture a medical image of the patient 185. In other embodiments, the camera 196 may receive image data from another source. For example, the camera 196 may receive image slices generated by the RF coil arrays transmitting the RF pulses. In some embodiments, the camera 196 may be a camera configured to record a live video feed within the imaging space 135 and display the live video feed via the user interface 190. The model 197 may detect imaging accessories on the patient 185 by analyzing the live video feed. In other implementations, the camera 196 may be configured to capture still images within the imaging space 135 and display the still image on the user interface 190. The model 197 may then detect imaging accessories on the patient 185 by analyzing the still image.

The machine learning model 197 (also referred to herein as “the model 197”) may be trained to detect different objects (e.g., imaging accessories, RF body coils 130, etc.) located on a patient 185 within the imaging space 135. In some embodiments, the machine learning model 197 may be a deep learning (DL) model trained to detect accessories. Specifically, the machine learning model 197 may be trained on images stored in an image library 198 such that the machine learning model 197 may detect new or different accessories without retraining the model. For example, in some embodiments, the image library 198 may be continually updated with new or additional images taken by the camera 196. The model 197 may be trained using template-based training. The combination of template-based training and continuous updating of the image library 198 may cause the model 197 to exhibit emergent behavior, meaning that the model 197 is able to detect objects that look different than the objects present in the images of the image library 198.

Generally, the model 197 is trained using template-based training to generate an emergent model. Specifically, the model 197 is trained using the images in the image library 198 to be able to detect, upon receipt of a medical image from the camera 196, whether an object (e.g., an accessory) is present in the medical image. As stated above, the model 197 may be trained once, and as the image library 198 is updated with additional images, object detection by the model 197 may improve.

The image library 198 may contain a plurality of images that include both images showing accessories and images not showing accessories. For example, the image library 198 may contain images that include objects that appear visually similar to accessories but are not actually accessories. Images showing or containing accessories may be stored as “accessory support set” images. Images now showing or containing accessories may be stored as “accessory mimic support set” images. The image library 198 may be stored locally on the medical imaging device or in a cloud-based networking system.

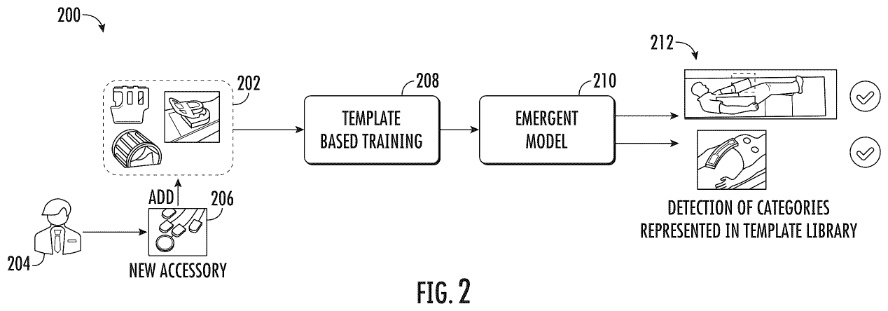

Referring now to FIG. 2, a flow diagram for a process 200 of object detection is shown, according to an example embodiment. The process 200 is describes a general, overall process of training the model 197 and performing object detection using the model 197.

As shown in process 200, the accessory library 202 may include a plurality of images of imaging accessories. The accessory library 202 may be similar to the image library 198. In some implementations of the process 200, a user or technician 204 of the medical imaging system 100 may add new accessories images 206 to the accessory library 202. For example, and as will be described in greater detail herein, when a technician 204 inspects a medical image that the model 197 has analyzed to detect objects within, the technician 204 may manually input an image of a detected (or undetected) object within the medical image to the accessory library 202. The model 197 may use the newly added images to improve future detection of objects in medical images.

At process 208, using the images in the accessory library 202 and/or the new accessory images 206, the model 197 is trained. Specifically, the model 197 may be trained using template-based training. That is, the model may be trained using images from the accessory library 202, as well as a training data set including annotated patient images showing imaging accessories, as well as annotated patient images showing objects visually similar to imaging accessories. The template-based training may cause the model 197 to be fully trained and function as an emergent model 210. The emergent model 210 may be the same as or similar to the model 197. An emergent model 210 may be one that is able to detect objects, specifically imaging accessories, that look visually different than the imaging accessories present in images of the accessory library 202.

At process 212, the trained emergent model 210 detects objects (e.g., imaging accessories) in various medical images. Specifically, the trained emergent model 210 analyzes medical images captured by the camera 196 of the image processing circuit 195. For example, when a patient 185 enters the imaging space 135, the camera 196 captures a medical image of the patient. The trained model 197 analyzes the medical image to detect whether an object (e.g., an imaging accessory) is present in the medical object based on images and categories of images stored in the accessory library 202. Specifically, the trained model 197 compares characteristics of the medical image to images of accessories in the accessory library 202. Upon a determination that a number of characteristics matching between the medical image and at least one image in the image library is at or above a predetermined threshold value, the trained model 197 identifies an object as being present in the medical image.

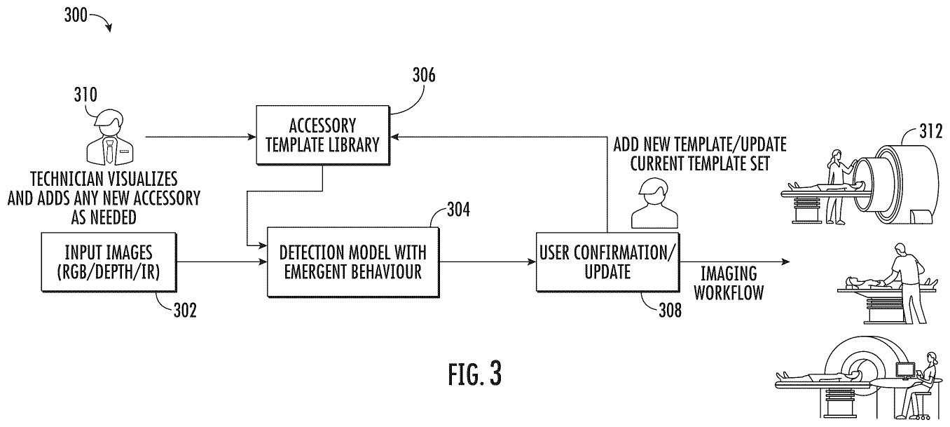

Referring now to FIG. 3, a flow diagram for a process 300 is shown, according to an example embodiment. The process 300 may describe an overall process or method for object detection using the model 197.

The process 300 begins with the model 197 (shown as the detection model 304) receiving one or more input images 302. In some implementations, input images 302 may be medical images of a patient 185 captured by the camera 196. The image processing circuit 195 (e.g., the model 197) may analyze the images and extract various features of the images perform object detection. For example, the model 197 may extract red, green, blue (RGB) values for each pixel of each medical image, depth information of each medical image, infrared (IR) information of each medical image, etc.

The input images are used as inputs to the model 304. The model 304 may be the same as or similar to the model 197, the emergent model 210, etc. In various embodiments, the model 304 may be a detection model characterized by emergent behavior. In various embodiments, images stored in the accessory template library 306 may also be used as inputs to the detection model 304. As stated herein, the model 304 compares the images stored in the accessory template library 306 with the input images 302 to detect the presence (or, in some examples, absence) of one or more accessories or objects in the input images 302. For example, the model 304 compares images stored in the accessory template library 306 that have been previously correctly identified as having accessories present or absent in the images with medical images that have not had objects/accessories correctly identified. The model 304 may perform the object detection in various ways. For example, the model 304 can perform object detection using a combination of enumeration of imaging accessories present and the detection of each accessory, multi-instance segmentation, classification using a saliency map, etc.

The model 304 outputs (e.g., to the user interface 190 for viewing by a technician) an indication of one or more detected objects (and/or an indication that no objects were detected) in the medical image. The model 304 may output the medical image with one or more objects detected by the model 304 by way of an overlaid object identifier (e.g., highlighting, boxing, or other indicators/identifiers).

At process 308, a user or technician confirms the outputs of the model 304. For example, a technician reviews the output image with the overlaid object identifiers to confirm that the model 304 has correctly identified all objects present in the medical image. In some implementations, the model 304 may incorrectly identify one or more objects in the medical image. For example, in some embodiments, the model 304 may detect an object and indicate that an object/accessory is present in the medical image when, in actuality, no object is present. This may be referred to as a false positive identification. In some embodiments, the model 304 may not detect an object as being present in the medical image when, in actuality, an object is present. This may be referred to as a false negative identification. The technician may review the output images and mark or flag any false positives, false negatives, true positives (e.g., objects the model 304 correctly identifies as being present in the medical image), and true negatives (e.g., objects the model 304 correctly identifies as being absent in the medical image). The reviewed images may be input to the accessory template library 306 and may be used in subsequent object detection by the model 304, thereby leading to improved object detection because the model 304 now has additional data against which to compare new input medical images.

Further, in some implementations, at process 310, a technician may input additional images to the accessory template library 306 that are not necessarily marked or flagged medical images that have been analyzed by the model 304 and reviewed by a technician. For example, at process 310, a user may input unrelated images including objects/imaging accessories to improve later object detection by the model 304. Additionally, at process 312, responsive to confirmation of the output medical image at process 308, imaging workflow may proceed. For example, responsive to correct identification of one or more objects/imaging accessories present in a medical image of a patient, an image (e.g., an MR image) of the patient may proceed.

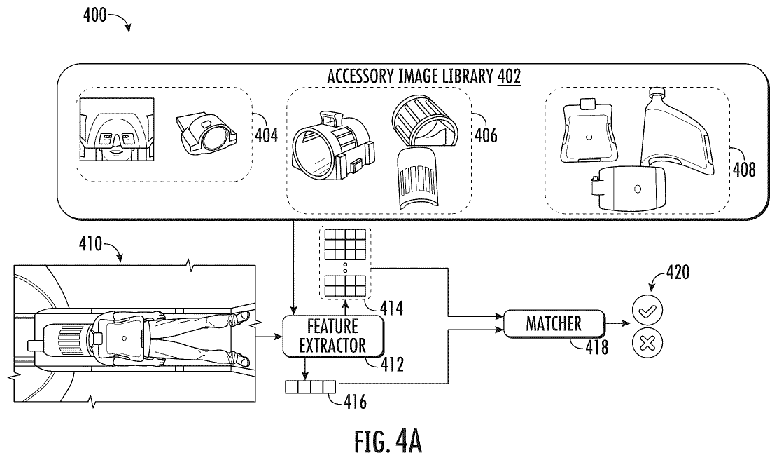

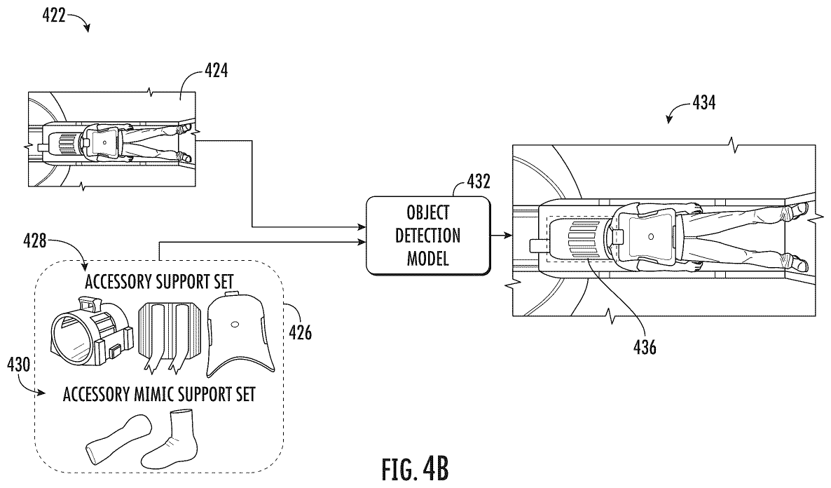

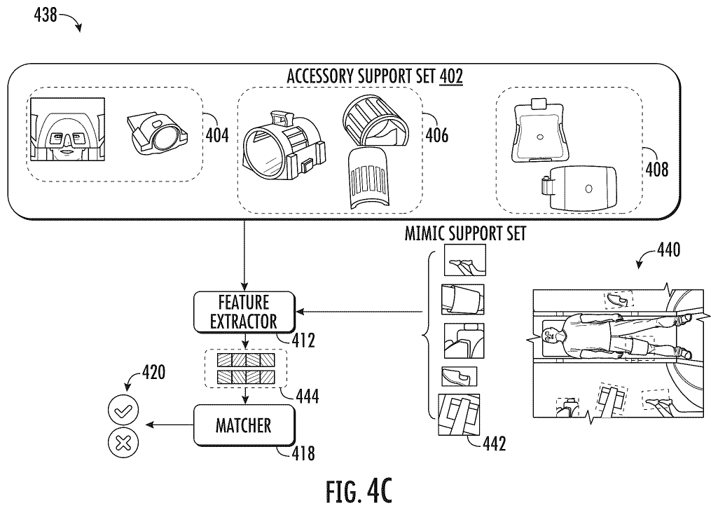

Referring now to FIG. 4A, a flow diagram for a process 400 is shown, according to an example embodiment. The process 400 may be a first flow diagram of a first step of a method of object detection. In some embodiments, the process 400 may be performed independently of any other method or process. The process 400 may describe a preprocessing method for template-based object detection using the model 197.

As shown, an accessory support set 402 stores a plurality of types of images. The accessory support set 402 may store images similar to those stored in the image library 198 and/or the accessory template library 306. Specifically, the accessory support set 402 may store a plurality of images of imaging accessories that have been positively identified (e.g., the images actually show imaging accessories). The accessory support set 402 may include a plurality of subsets of image types. For example, the accessory support set 402 may include images of a plurality of types of imaging accessories, and images containing different imaging accessories may be sorted by imaging accessory type. For example, a first image subset 404 may include images of a first type of accessory, a second image subset 406 may include images of a second type of accessory, and a third image subset 404 may include images of a third type of accessory.

The process 400 includes an input image 410 being input to a feature extractor 412. As shown, the input image includes a patient 185 with various types of imaging accessories placed on/around the patient. Images from the accessory support set 402 are also input to the feature extractor 412. The feature extractor 412 extracts, from the input image and from at least one image from the accessory support set 402, one or more elements or features of the images. For example, as described in FIG. 3, the feature extractor 412 may extract RGB values, depth values, and/or IR values from each image.

The feature extractor 412 outputs a template image feature set 414 and an input image feature set 416. The template image feature set 414 may include extracted image values from the at least one image from the accessory support set 402, and the input image feature set 416 may include extracted image values from the at least one input image 410.

The template image feature set 414 and the input image feature set 416 are input to a matcher 418. The matcher 418 compares the features in the template image feature set 414 with the features in the input image feature set 416. For example, the matcher 418 may determine a similarity match between the feature set 414 and the feature set 416. Determining a similarity match may include identifying a number of matches between the extracted features and the features of the library image. At process 420, responsive to determining that the similarity score and/or a number of matches is at or above a threshold value, the matcher 418 determines that the detected features in the input image match features in a template image and that the detected object(s) in the input image is or are imaging accessories. Responsive to determining that the similarity score and/or a number of matches is below a threshold value, the matcher 418 determines that the detected features in the input image do not match features in a template image and that the detected object(s) in the input image is or are not imaging accessories. In some embodiments, determining that the similarity score and/or a number of matches is below a threshold value may indicate that the feature extractor 412/model 197 did not detect any imaging accessory/object in the input image.

Referring now to FIG. 4B, a process 422 illustrating a first step of a method of object detection is shown, according to an example embodiment. In some embodiments, the process 422 depicts a method or process of training an object detection model with emergent behaviors. For example, the object detection model may be the same as or similar to the model 197. In some embodiments, the process 422 may be similar to the process 400 described with respect to FIG. 4A (e.g., include one or more similar steps or processes).

The process 400, begins when a test image 424 is input to an object detection model 432. In various implementations, stored reference images may also be input to the object detection model 432. For example, the accessory image library 426 may include images classified as belonging to or including an accessory support set 428 or an accessory mimic support set 430. The accessory support set 428 may include images of accessories that have previously been positively identified (e.g., by the model 432 and/or a technician) as being imaging accessories. The accessory mimic support set 430 may include images of objects that are not imaging accessories but look similar (e.g., may have similar extracted features) to imaging accessories. The object detection model 432 may use the accessory support set 428 and accessory mimic support set 430 information to analyze the test image 424. For example, the object detection model 432 may analyze the test image and images from the accessory support set 428 and accessory mimic support set 430 (e.g., by using feature extraction as described with respect to FIG. 4A).

Responsive to a determination that one or more features of the test image 424 match one or more features of images in the accessory support set 428, the object detection model 432 may determine that the test image 424 includes one or more imaging accessories. Responsive to a determination that one or more features of the test image 424 match one or more features of images in the accessory mimic support set 430, the object detection model 432 may determine that the test image 424 does not include imaging accessories.

The object detection model 432 may output an image 434 with an object detection marker 436 overlaid on the image 434. In various embodiments, the image 434 may be the same as the test image 424 with one or more object detection markers 436 included (e.g., when the object detection model 432 determines that an object is present in the image). In various implementations, the object detection marker 436 may include an indication of a confidence level of the detection. For example, based on the comparisons between the test image 424 and the accessory images, the object detection model 432 may determine a confidence level or other indication of how confident the model is that an object has been correctly identified in the test image 424. The confidence level may be expressed as a decimal, a percentage, a fraction, etc. The confidence level may be displayed on the output image 434 proximate the object detection marker 436.

Referring now to FIG. 4C, a process 438 for post processing object detection results is shown, according to an example embodiment. The process 438 may be used to filter out false positive results. The process 438 may include similar components and/or processes as the process 400 described with respect to FIG. 4A, such as, for example, the accessory support set 402 containing first, second, and third accessory types 404-408, the feature extractor 412, and the matcher 418. In some embodiments, the process 438 may be similar to the process 400 described with respect to FIG. 4A and/or the process 422 described with respect to FIG. 4B (e.g., include one or more similar steps or processes).

Similar to process 400, an input image 410 is input to the feature extractor 412, as well as images from the accessory support set 402. A plurality of images belonging to an accessory mimic support set 442 may also be input to the feature extractor 412. The images belonging to the accessory mimic support set 442 may be images of objects that look similar to imaging accessories but are not actually imaging accessories.

The feature extractor 412 may extract features (e.g., RGB values, depth information, IR information, etc.) from each of the image 410, the image(s) from the accessory support set 402, and the image(s) from the accessory mimic support set 442. Th extracted features 444 may be compared with one another by the matcher 418. For example, the matcher 418 may compare the extracted features of the image 410 with the extracted features of each of the images from the set 402 and the set 442 and generate similarity scores. At process 420, the matcher 418 may indicate or detect objects (e.g., image accessories) in the image 410.

The image 440 may be a resulting image generated by the machine learning model (e.g., the model 197) responsive to the matcher 418 determining similarity scores between the image 410 and the image sets 402 and 442. For example, as shown in FIG. 4C, the image 440 may include object identifiers overlaid on both properly identified objects (e.g., actual imaging accessories) and improperly identified objects (e.g., not imaging accessories). Object identifiers overload on improperly identified objects may indicate that a false positive has occurred. The inclusion of the accessory mimic support set images may help the model (e.g., the feature extractor 412 and the matcher 418) to identify and filter out false positives. For example, when the feature extractor 412 compares features of the image 410 with features of the images from the mimic set 442, a similarity score at or above a threshold value may indicate that the object is not an image accessory and should not be identified as such. If the image 410 was not compared to the mimic set 442 and was only compared to the accessory support set 402, the similarity score between the features may still be at or above a threshold value such that the matcher 418 generates a false positive result.

Conversely, in various embodiments, when the feature extractor 412 generates similarity scores between both the image 410 and the accessory support set 402 and the image 410 and the mimic support set 442 that are at or above a threshold value, the matcher 418 may classify an object based on which similarity score is higher. For example, the threshold value may be 0.75, the similarity score between the image 410 and the accessory support set 402 may be 0.8, and the similarity score between the image 410 and the mimic support set 442 may be 0.9. The matcher 418 may identify the larger similarity score and determine that the object in the image 410 is not an image accessory and should not be detected/classified as such.

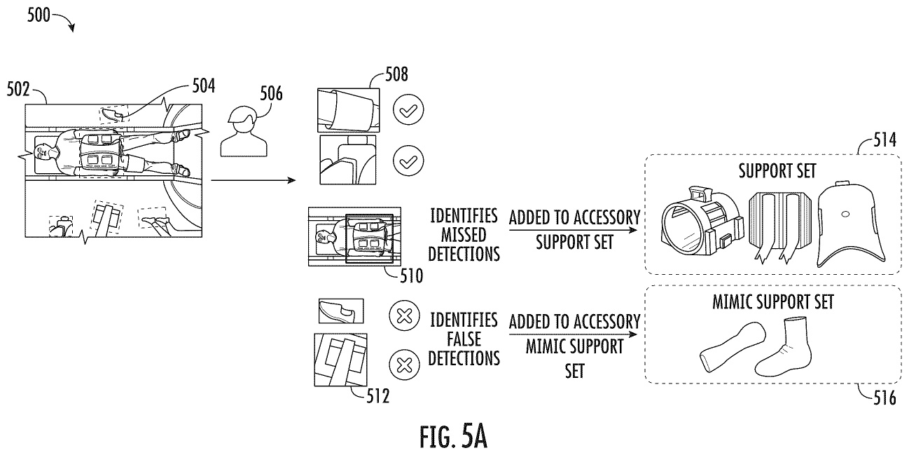

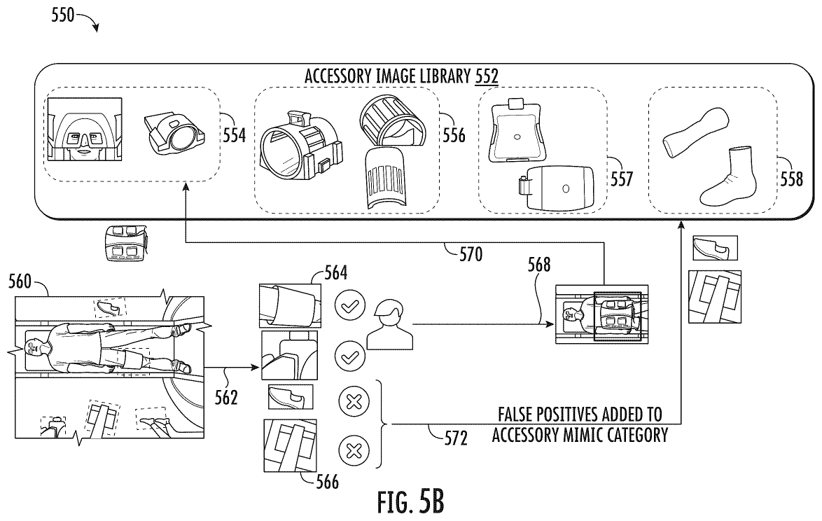

Referring now to FIG. 5A, a process 500 for receiving user feedback and/or updating an image library (and/or accessory support sets and/or mimic support sets) is shown, according to an example embodiment. The processes described with respect to FIGS. 5A and 5B may be performed subsequent to the processes described with respect to FIGS. 4A through 4C. For example, the processes in FIGS. 4A through 4C describe a process of training the model and detecting (and/or not detecting) objects or imaging accessories in an image. The processes described with respect to FIGS. 5A and 5B may occur after the objects have been detected.

The process 500 may begin with providing an image 502 to a technician 506. The image 502 may be the same as the output image 440 of FIG. 4C (e.g., the input image with overlaid object identifiers 504 indicating objects the model has identified as imaging accessories). The technician 506 may view the image 502 (e.g., via the user interface 190) and the overlaid object identifiers 504. Specifically, the technician may review the placement of the object identifiers 504 to confirm that all objects in the image 502 have been identified (as indicated by an object identifier 504 overlaid on the object) and that no objects have been improperly identified (as indicated by an object identifier 504 overlaid on an object that is not an imaging accessory).

At process 508, the technician 506 may confirm that the model has properly identified an object/image accessory. The technician may confirm identification of an object by interacting with the user interface 190. For example, in some embodiments, the user interface 190 may be a touchscreen device displaying the image 502. The technician 506 may gesture, click, or otherwise interact with the user interface 190 to, for example, select an object identifier 504. The technician 506 can acknowledge the proper detection of the object. For example, responsive to selecting an object identifier 504, the user interface 190 may display a menu or other icon allowing the technician 506 to approve or confirm placement of the object identifier 504 and proper detection of the object/imaging accessory. In some embodiments, the technician 506 may be able to manipulate the size or dimensions of an object identifier 504 to ensure the object identifier 504 is accurately placed and sized on an imaging accessory.

At process 510, the technician 506 may identify objects/imaging accessories within the image 502 that were not identified by the model (e.g., the technician 506 may correct false negatives by the model). The technician 506 may interact with the user interface 190 to draw, outline, or otherwise create or overlay an object identifier 504 on an imaging accessory that was not identified by the model but should have been. The object identifier 504 created by the technician 506 may indicate a portion of the image 502 (e.g., the portion including the object/imaging accessory) that is to be added to the image library 198. Specifically, the portion of the image 502 is to be added to an accessory support set 514 that may be the same as or similar to the accessory support set 402. The model 197 may extract the portion of the image 502 defined by the object identifier 504 and transmit the portion of the image 502 to the accessory support set 514.

At process 512, the technician 506 may identify object identifiers 504 placed on the image 502 that have incorrectly identified a non-imaging accessory object as an imaging accessory/object (e.g., the technician 506 may correct false positives by the model). The technician 506 may interact with the user interface 190 to select an incorrectly generated object identifier 504. Responsive to selection of the incorrectly generated object identifier 504, the user interface 190 may display a menu or other icon that is selectable by the technician 506 to deny or remove placement of the object identifier 504. The incorrectly generated object identifier 504 may indicate a portion of the image 502 (e.g., the portion including the incorrectly identified object) that is to be added to the image library 198. Specifically, the portion of the image 502 is to be added to an accessory mimic support set 516 that may be the same as or similar to the accessory support set 402. The model 197 may extract the portion of the image 502 defined by the object identifier 504 and transmit the portion of the image 502 to the accessory mimic support set 516.

Referring now to FIG. 5B, a process 550 for receiving user/technician feedback on object detection performed by the model 197 and subsequently updating the image library 198 is shown, according to an example embodiment. The process 550 may be similar to the process 500 described above with respect to FIG. 5A. The process 550 may be performed subsequent to any of the processes 400, 422, or 438 described above with respect to FIGS. 4A-4C. As shown in FIG. 5B, an accessory image library 552 may include a plurality of image categories, including a first image category 554 that stores images of objects belonging to a first type of category of imaging accessory, a second image category 556 that stores images of objects belonging to a second type of category of imaging accessory, a third image category 557 that stores images of objects belonging to a third type of category of imaging accessory, and an accessory mimic set 558 that stores images of objects that are not imaging accessories.

At process 562, an image 560 (that is the same as or similar to the test image 502) that has been analyzed by the model 197 (e.g., object detection has been performed) may be viewed by a technician. Images 564 indicate objects present in the image 560 that were not identified as imaging accessories by the model 197 (e.g., false negatives). Images 566 may indicate objects present in the image 560 that were identified as imaging accessories by the model 197 but are not actually imaging accessories (e.g., false positives).

At process 568, the technician marks the objects in the image 560 that were not detected by the model 197. In some embodiments, the technician may also identify a category or type of the object that was not detected. At process 570, the false negatives (e.g., the images 564) may be added to the accessory image library 552. Specifically, each image 564 may be added to an image set within the accessory image library 552 corresponding to the object category identified by the technician. For example, the technician may indicate that an image 564 is of an object belonging to a first type of accessory. The model 197 may add the image 564 to the first accessory category 554.

At process 572, the technician may identify the false positive images 566 and may cause the false positive images 566 to be added to the accessory image library 552. Specifically, the false positive images 566 may be added to an accessory mimic image set 558 within the accessory image library 552. In some embodiments, the technician does not identify the false positive images 566 and the model 197 automatically adds the false positive images 566 to the accessory mimic image set 558.

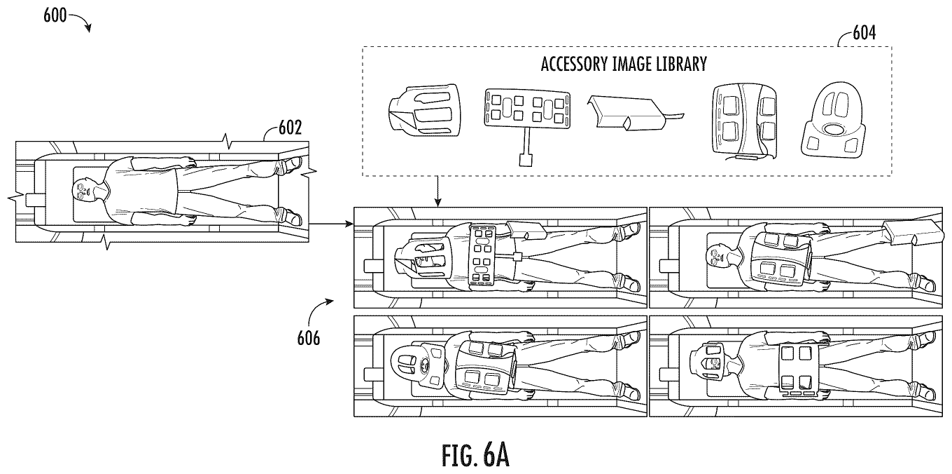

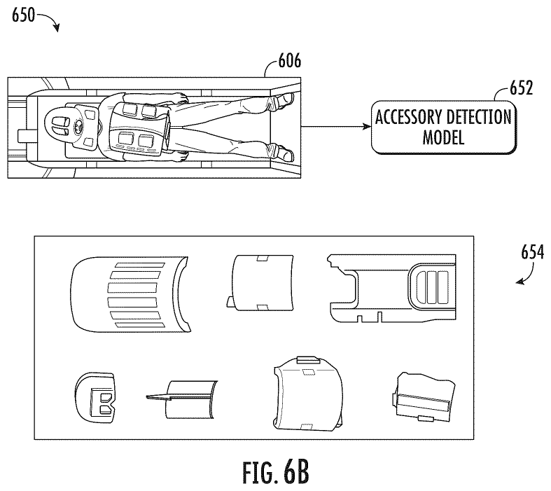

Referring now to FIG. 6A, a process 600 for training a model to perform object detection is shown, according to an example embodiment. The process 600 may describe a first step or portion of an overall process for training the model (e.g., the model 197). For example, the process 600 may describe a first step for training the model and process 650, which will be described in greater detail below with respect to FIG. 6B, may describe a second step for training the model. Specifically, the process 600 may describe a method of data simulation. The process 600 may be performed by the image processing circuit 195.

To simulate data, an image 602 is taken of the patient 185 using, for example, the camera 196. The image 602 may be of the patient 185 with no imaging accessories present. An accessory image library 604, which may be the same as or similar to the image library 198, the accessory support set 402, etc., may include a plurality of images of imaging accessories. The model 197 may superimpose or overlay different images of imaging accessories from the accessory image library 604 onto the image 602. Resulting images 606 are shown in FIG. 6A. The data simulation process 600 may cause various types of images 606 to be generated. For example, images 606 may be created that show various types of imaging accessories overlaid on the patient 185. For example, imaging accessories of different color jitter, rotation, translation, orientation, contrast, shape, size, etc. may be overlaid onto the image 602 to create variety. The various images 606 may be used to train the model 197 to be able to identify different imaging accessories having different appearances.

Referring now to FIG. 6B, a process 650 for training a model to perform object detection is shown, according to an example embodiment. The process 650 my include training the model (e.g., the model 197) using the simulated data generated and performed at process 600. For example, the process 650 may include training the model 197 on images 606 generated at process 600.

During process 650, a simulated image 606 is input to an accessory detection model 652. The accessory detection model 652 may be the same as or similar to the model 197. The accessories 654 illustrate various types of accessories that may be included in an image 606 for use in training the accessory detection model 652. In various embodiments, the accessory detection mode 652 may be trained using any of the methods described herein. For example, the accessory detection model 652 may be trained using the methods described with respect to FIG. 2.

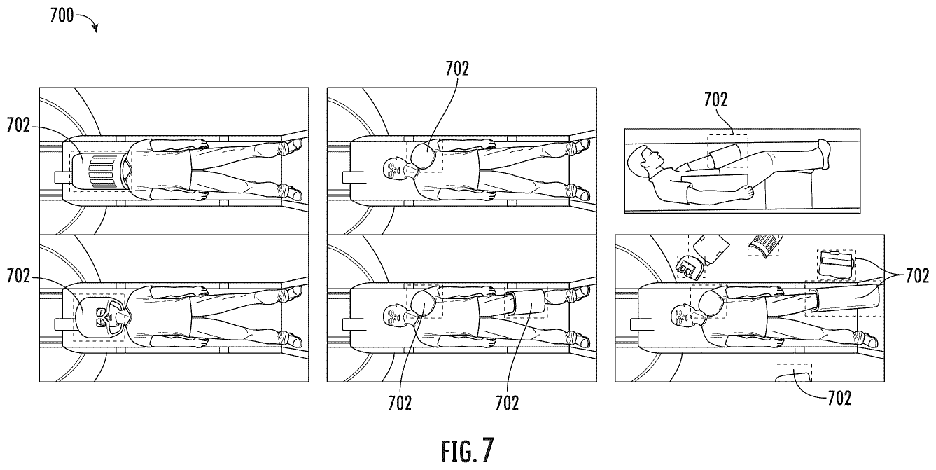

Referring now to FIG. 7, a plurality of test images 700 are shown, according to an example embodiment. The test images 700 each include one or more imaging accessories 702 overlaid on the patient present in the image 700. The images 700 may be used by the model 197 as training data. For example, the model 197 may be trained using the images 700, and the model 197 may learn to make inferences about the presence and absence of imaging accessories in previously unseen images based on analysis of the images 700.

In some embodiments, the model 197 may be trained make inferences/detect objects in various environments different than those present in the images 700. For example, upon being trained on the images 700, the model 197 may be able to detect imaging accessories present in images of new or different patients or test subjects, new or different environments, etc. For example, the images 700 may show a male patient in a supine position in a hospital room and an imaging accessory on the leg. The model 197 may use its training on the images 700 to be able to detect an imaging accessory on the arm of a female patient in a lateral recumbent position in an imaging suite.

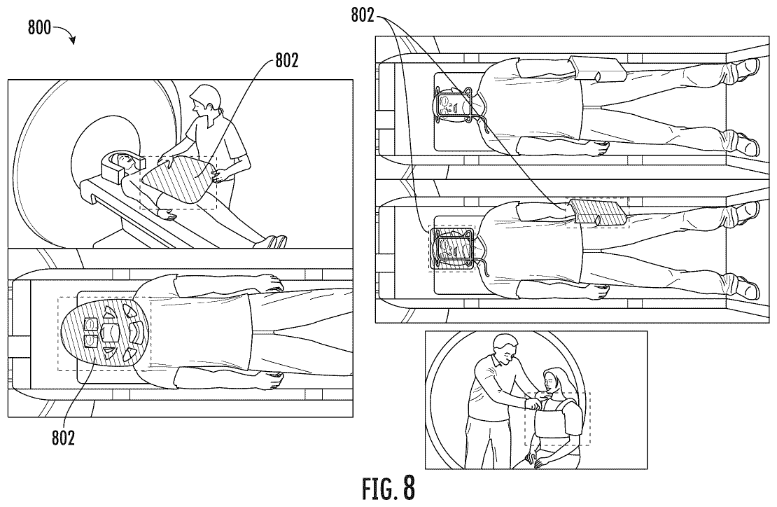

Referring now to FIG. 8, a plurality of images 800 are shown with object identifiers 802, according to an example embodiment. As stated above with respect to FIG. 7, the model 197 may be able to detect imaging accessories present in images different than those used to train the model 197. Specifically, as shown in FIG. 8, the model 197 may be able to correctly identify objects in various types of images. For example, the model 197 may be able to perform object detection on images collected from marketing brochures, advertisements, etc., that show imaging accessories having unusual or different designs, shapes, setups, etc. As such, the model 197 may demonstrate emergent behaviors and generalize the simulated data to be able to detect various kinds and types of imaging accessories.

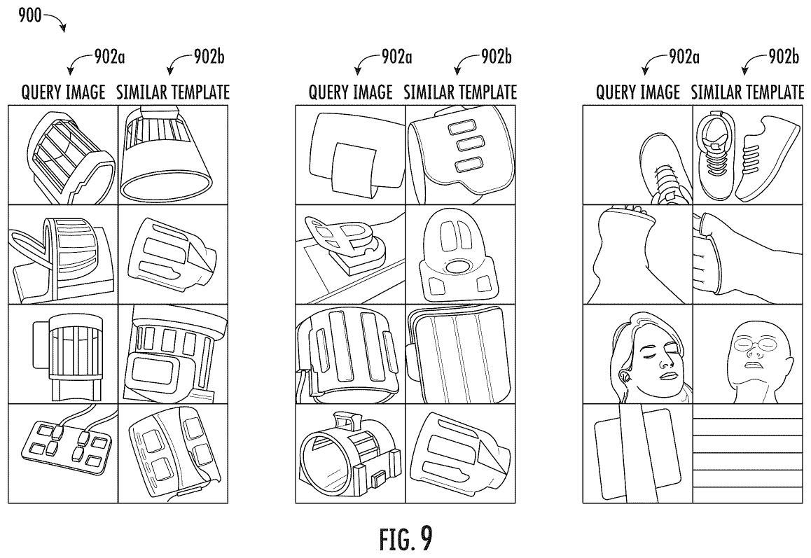

Referring now to FIG. 9, a plurality of image sets 900 are shown, according to an example embodiment. As shown, FIG. 9 includes a plurality of query images 902a and a plurality of corresponding template images 902b.

The query images 902a may be or include imaging accessories present in images input to the model 197 that are to have object detection performed. That is, the query images 902a may not be training data. The model 197 may receive one or more query images 902a and may search the image library 198 to retrieve a stored template image 902b that is similar to a query image 902a. For example, the template images 902b may be stored in the image library 198. When the model 197 receives a query image 902a, the model 197 may perform feature extraction or another method to determine a template image 902b most similar to the query image 902a (e.g., a query image and template image pair having a highest similarity score).

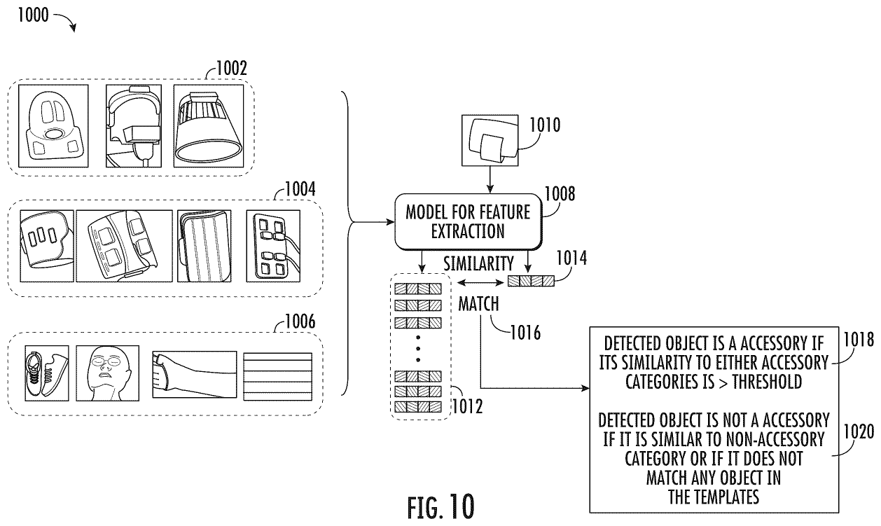

Referring now to FIG. 10, a process 1000 for template-based matching is shown, according to an example embodiment. Process 1000 may be performed subsequent to one or more of the process 600 or 650described with respect to FIGS. 6A and 6B. For example, responsive to training the model at process 650, at process 1000, one or more templates may be used to match detected objects with stored image data. The process 1000 may be used to remove or reduce instances of false positives by the model 197.

As shown, a plurality of image types 1002, 1004, and 1006 may be input into a model 1008 for feature extraction. In various embodiments, the image types 1002, 1004, and 1006, may be or include images of different types of imaging accessories. For example, the image type 1002 may be or include a subset of imaging accessories that are rigid (e.g., rigid coils). The image type 1004 may be or include a subset of imaging accessories that are flexible (e.g., flex coils). The image type 1006 may be or include a subset of images of non-imaging accessory objects. Particularly, images of the image type 1006 may be or include objects commonly mistaken for imaging accessories.

The image data from the image types 1002-1006 may be input into the model 1008 along with an image 1010 of a detected imaging accessory. The image 1010 may be captured by the camera 196. As described with respect to FIGS. 4A and 4C, the model 1008 may extract a plurality of features from each of the images from the image types 1002-1006, as well as the image 1010. The model 1008 may extract and determine a plurality of template features 1012 that correspond to images from the image types 1002-1006. The model 1008 may also extract and determine a plurality of test image features 1014 that correspond to the image 1010.

At process 1016, the model 1008 determines a similarity match between the test image features 1014 and the template features 1012. For example, the model 1008 may determine a number of extracted features that the images 1002-1006 and the image 1010 have in common. Responsive to determining the similarity match, at process 1018, the model 1008 may determine that the detected object in the image 1010 is an imaging accessory by determining that a similarity of the extracted features 1014 to the extracted features 1012 of the image type 1002 and/or the image type 1004 is greater than or equal to a threshold value. That is, the model 1008 may classify an object in an image 1010 as an imaging accessory when features in the image 1010 match features in images corresponding to a first and/or second imaging accessory type.

Responsive to determining the similarity match, at process 1020, the model 1008 may determine that the detected object in the image 1010 is not an imaging accessory by determining that a similarity of the extracted features 1014 to the extracted features 1012 of the image type 1006 is greater than or equal to a threshold value. That is, the model 1008 may classify an object in an image 1010 as not being an imaging accessory when features in the image 1010 match features in images that have been previously identified as not being imaging accessories. Additionally or alternatively, the model 1008 may determine that a detected object in the image 1010 is not an imaging accessory by determining that extracted features 1014 do not match any of images of any image type 1002-1006. That is, when the similarity score between the features 1014 of the image 1010 and any of the template features 1012 is below a threshold value, the model 1008 may classify the image 1010 as not including any objects and/or may classify a detected object in the image 1010 as not being an imaging accessory.

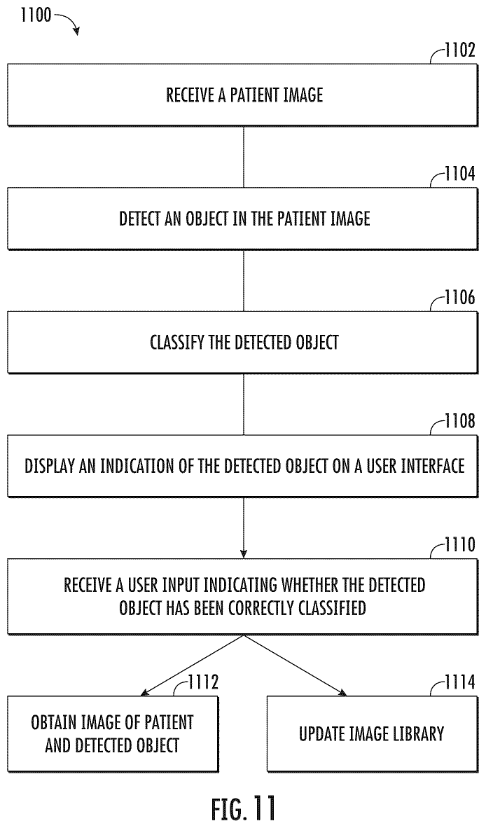

Referring now to FIG. 11, a method 1100 is shown for detecting objects in a medical imaging system, according to an example embodiment. Specifically, the method 1100 may be used to detect imaging accessories in patient images.

At process 1102, the image processing circuit 195 receives, via a camera of the medical imaging device (e.g., the camera 196), a patient image of a patient within an imaging space. For example, the image processing circuit 195 may be part of the medical imaging system 100, which may include a medical imaging device configured to transmit electromagnetic pulse signals to a patient within the imaging space. In some embodiments, the medical imaging device is a magnetic resonance (MR) imaging device. In various embodiments, a machine learning model (e.g., the model 197) may receive the patient image from the camera 196.

In some embodiments, the image processing circuit 195 includes an image library (e.g., the image library 198) that includes a plurality of library images depicting known objects. In some embodiments, the image library comprises a first set of images and a second set of images, the first set of images comprising images previously identified as representing imaging accessories and the second set of images comprising images previously identified as not representing imaging accessories.

For example, the library images may be or include images belonging to an accessory support set and/or an accessory mimic support set. The images belonging to the accessory support set may be categorized based on a type of imaging accessory (e.g., rigid or flexible). In some embodiments, the image library is stored in a cloud computing network. In other embodiments, the image library is stored locally on the medical imaging device.

In some embodiments, the method 1100 may include training a model (e.g., the model 197) on the plurality of library images. The model may be trained to identify a known object present in the patient image. The model 197 may be an emergent model that is trained once before a deployment of the model.

At process 1104, the model 197 may detect an object in the patient image. The object may be or represent an imaging accessory (e.g., an MR coil, an ECG lead, etc.). At process 1106, the model 197 may classify the detected object as either one of a known object from the image library or an unknown object.

In some embodiments, the detected object is classified, by the model 197, as a known object from the image library 198 when a number of image characteristics matching between the detected object and at least one image from the image library 198 is at or above a threshold value. In some embodiments, the detected object is classified as an unknown object when a number of image characteristics matching between the detected object and at least one image from the image library is below a threshold value.

In some implementations, classifying the detected object includes extracting features from the detected object. Classifying the detected object may further include comparing the extracted features to features in one or more library images and determining a similarity match between the extracted features and the features of the library images. In some embodiments, determining the similarity match comprises identifying a number of matches between the extracted features and the features of the library images. Classifying the detected object may further include classifying the detected object as a known object from the image library responsive to determining that the similarity match is at or above a threshold value, and classifying the detected object as an unknown object responsive to determining that the similarity match is below the threshold value.

At process 1108, the model 197 may display an indication of the detected object on a user interface (e.g., the user interface 190) of the medical imaging device. For example, the model 197 may cause an object identifier to be overlaid on the detected object in the patient image.

At process 1110, the model 197 may receive, via the user interface 190, a user input indicating whether the detected object has been correctly classified as a known object (e.g., an imaging accessory) or an unknown object (e.g., an object similar to an imaging accessory but not an imaging accessory) by the model. For example, a technician may manually review the image classification performed by the model 197. The technician may confirm that the model has correctly identified an object, may indicate that the model failed to identify an object, and/or may indicate that the model incorrectly identified an object. Subsequent to process 1110, the image processing circuit 195 may perform one or both of the processes 1112 and 1114.

At process 1112, responsive to receiving the user input at process 1110, the medical imaging system 100 may obtain a medical image of the patient and the detected object. For example, the medical imaging system 100 may obtain an MR image of the patient.

At process 1114, responsive to receiving the user input at process 1110, the model 197 may update the image library based on the user input. For example, the model 197 may update the image library 198 with images the technician indicated as not being identified by the model 197 and/or indicated as being incorrectly identified by the model.

In some embodiments, the detected object of processes 1102-1114 is a first object and the user input is a first user input. The method 1100 may further include receiving a second user input identifying a second object that the model did not detect in the patient image and updating the image library based on the second user input.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. A medical imaging system comprising:
a medical imaging device configured to transmit electromagnetic pulse signals to a patient within an imaging space having a magnetostatic field formed therein to obtain medical images of the patient;
an image library comprising a plurality of library images depicting known objects; and
a processing circuit having a processor coupled to a memory device storing instructions thereon that, when executed, cause the processing circuit to:
receive, via a camera of the medical imaging device, a patient image of the patient within the imaging space;
detect, using a trained model, an object in the patient image;
classify, using the trained model, the detected object as either one of a known object from the image library or an unknown object;
display an indication of the detected object on a user interface of the medical imaging device;
receive, via the user interface, a user input indicating whether the detected object has been correctly classified as a known object or an unknown object by the trained model; and
at least one of obtain a medical image of the patient and the detected object or update the image library based on the user input.

2. The system of claim 1, wherein the model is an emergent model trained once before a deployment of the model.

3. The system of claim 1, wherein the object is a first object, the user input is a first user input, and the instructions, when executed, further cause the processing circuit to:
receive a second user input identifying a second object that the model did not detect in the patient image; and
update the image library based on the second user input.

4. The system of claim 3, wherein the image library comprises a first set of images and a second set of images, the first set of images comprising images previously identified as representing imaging accessories and the second set of images comprising images visually similar to imaging accessories but identified as not representing imaging accessories.

5. The system of claim 1, where the detected object is classified as a known object from the image library when a number of image characteristics matching between the detected object and at least one image from the image library is at or above a threshold value, and where the detected object is classified as an unknown object when a number of image characteristics matching between the detected object and at least one image from the image library is below a threshold value.

6. The system of claim 1, wherein classifying each representation comprises:
extracting features from the detected object;
comparing the extracted features to features in one or more library images;
determining a similarity match between the extracted features and the features of the library images;
classifying the detected object as a known object from the image library responsive to determining that the similarity match is at or above a threshold value; and
classifying the representation as an unknown object responsive to determining that the similarity match is below the threshold value.

7. The system of claim 6, wherein determining the similarity match comprises identifying a number of matches between the extracted features and the features of the library images.

8. The system of claim 1, wherein the image library is stored in a cloud computing network.

9. The system of claim 1, wherein the image library is stored locally on the medical imaging device.

10. The system of claim 1, wherein the medical imaging device is one of a magnetic resonance (MR) imaging device, a computed tomography (CT) imaging device, a positron emission tomography (PET) imaging device, a single-photon emission computed tomography (SPECT) imaging device, or an X-ray imaging device.

11. The system of claim 1, wherein the instructions further cause the processing circuit to train a model on the plurality of library images, the model trained to identify a known object present in the patient image.

12. A medical imaging system comprising:
a medical imaging device configured to transmit electromagnetic pulse signals to a patient within an imaging space having a magnetostatic field formed therein to obtain medical images of the patient;
an image library comprising a plurality of library images depicting known objects;
a camera associated with the medical imaging device, configured to receive a patient image of a patient within the imaging space;
a trained model configured to:
detect an object in the patient image; and
classify the detected object as either one of a known object from the image library or an unknown object; and
a user interface configured to:
display an indication of the detected object; and
receive a user input indicating whether the detected object has been correctly classified as a known object or an unknown object by the trained model;
wherein the system is configured to at least one of obtain a medical image of the patient and the detected object or update the image library based on the user input.

13. The system of claim 12, wherein the object is a first object, the user input is a first user input, and the system is further configured to:
receive a second user input identifying a second object that the model did not detect in the patient image; and
update the image library based on the second user input.

14. The system of claim 12, wherein the trained model is configured to classify each representation by:
extracting features from the detected object;
comparing the extracted features to features in one or more library images;
determining a similarity match between the extracted features and the features of the library images;
classifying the detected object as a known object from the image library responsive to determining that the similarity match is at or above a threshold value; and
classifying the representation as an unknown object responsive to determining that the similarity match is below the threshold value.

15. The system of claim 14, wherein determining the similarity match comprises identifying a number of matches between the extracted features and the features of the library images.

16. The system of claim 12, wherein the system is configured to train the model on the plurality of library images, the model trained to identify a known object present in the patient image.

17. A method comprising:
receiving, by a processing circuit, via a camera of a medical imaging device, a patient image of a patient within an imaging space;
detecting, by the processing circuit, using a trained model, an object in the patient image;
classifying, by the processing circuit, using the trained model, the detected object as either one of a known object from an image library or an unknown object;
displaying, by the processing circuit, an indication of the detected object on a user interface of the medical imaging device;
receiving, by the processing circuit, via the user interface, a user input indicating whether the detected object has been correctly classified as a known object or an unknown object by the trained model; and
at least one of obtaining, by the processing circuit, a medical image of the patient and the detected object or updating, by the processing circuit, the image library based on the user input.

18. The method of claim 17, wherein the object is a first object, the user input is a first user input, and the method further comprises:
receiving, by the processing circuit, a second user input identifying a second object that the model did not detect in the patient image; and
updating the image library based on the second user input.

19. The method of claim 17, where the detected object is classified as a known object from the image library when a number of image characteristics matching between the detected object and at least one image from the image library is at or above a threshold value, and where the detected object is classified as an unknown object when a number of image characteristics matching between the detected object and at least one image from the image library is below a threshold value.

20. The method of claim 17, wherein classifying each representation comprises:
extracting features from the detected object;
comparing the extracted features to features in one or more library images;
determining a similarity match between the extracted features and the features of the library images;
classifying the detected object as a known object from the image library responsive to determining that the similarity match is at or above a threshold value; and
classifying the representation as an unknown object responsive to determining that the similarity match is below the threshold value.
